(19) United States
(12) Patent Application Publication
(10) Pub. No.: US 2023/0355097 A1
(43) Pub. Date: Nov. 9, 2023

(54) Apparatus and process for electromagnetic imaging

(71) Applicant: EMvision Medical Devices Ltd, Brisbane QLD (AU)

(72) Inventors: Aida Brankovic, Brisbane QLD (AU); Amin Abbosh, Brisbane QLD (AU)

(21) Appl. No.: 18/026,227

(22) PCT Filed: Sep. 14, 2021
(86) PCT No.: PCT/AU2021/051058

§ 371 Date: Mar. 14, 2023

(30) Foreign Application Priority Data
Sep. 14, 2020 (AU) 2020903286

(51) Int. Cl. A61B 5/00; A61B 5/055

(52) U.S. Cl. CPC A61B 5/0042; A61B 5/7264; A61B 5/055; A61B 2576/026

(57) ABSTRACT

A computer-implemented process for electromagnetic imaging, the process including the steps of: accessing scattering data representing at least a two-dimensional array of measurements of electromagnetic wave scattering by internal features of an object, wherein the object is generally symmetrical with respect to a plane of symmetry through the object, and each said measurement represents scattering of electromagnetic waves emitted by a corresponding antenna of an array of antennas disposed about the object as measured by a corresponding antenna of the array of antennas; and processing the scattering data to generate image data representing a spatial distribution of at least one internal feature of the object, wherein the generation of the image data does not involve tomographic reconstruction but is in accordance with statistical metrics of similarity between pairs of corresponding regions within the object on either side of the plane of symmetry.

### TECHNICAL FIELD

The present invention relates to electromagnetic imaging or characterisation, and in particular to an apparatus and process for electromagnetic imaging.

### BACKGROUND

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Whilst magnetic resonance imaging (MRI) and computed tomography (CT) are gold standard medical imaging modalities, they are very expensive, limited in number for a given community, bulky and non-portable for emergency situations, and take a very long time (typically up to about 40 min) to prepare and scan the patient. Accordingly, electromagnetic based imaging, localization and classification of stroke and other pathologies has been widely studied in the literature as a much more affordable, readily available and portable imaging alternative. Low-power electromagnetic based imaging (at frequencies from 100 MHz, and typically up to no more than 4 GHz) is of particular interest because the shorter wavelength electromagnetic fields can penetrate further into the human head and produce images with higher spatial resolution than electromagnetic fields with frequencies below 100 MHz.

Research studies are performed utilizing antenna arrays, wherein each antenna has a corresponding dedicated and independent electronic transmit-receive channel to enable the collection of an entire matrix of measured scattering parameters, typically but not always being S-parameters or Z-parameters. For example, for each frequency point in a spectrum of frequencies, the Su and Su-parameters can be directly collected by a vector network analyzer and stored as a 2-dimensional N×N matrix, where N is the number of channels (and thus antennas) in the array. In the remainder of this specification, S-parameter measurements are used as representative examples of scattering parameters, although it should be understood that other types of electromagnetic scattering measurements known to those skilled in the art, such as Z-parameters for example, can be used instead of or in addition to S-parameters.

The antennas can be wide and varied in configuration and style, for instance often taking the form of dielectrically loaded waveguides or patch antennas. The size of the antennas determines both the number of antennas that can be fitted around the head or other body part, as well as the frequency bandwidth over which the antennas are able to operate. For example, in the case of the human head imaging, typically the antennas are arranged circumferentially around the head, with each antenna directed inwards towards the head. Normally, a coupling medium is inserted between the antenna apertures and the head surface in order to reduce the impedance mismatch and power reflection.

Ideally, S-parameter N×N matrix measurements are first performed with the target pathology (such as stroke), and then the measurements are repeated with a background reference that does not contain the target pathology. This background reference can, for example, involve any one or combination of the following: (i) an empty space background reference, (ii) a homogeneous reference such as a water bath, (iii) a dedicated phantom that fills the space within the array, (iv) a dedicated homogenous or inhomogeneous phantom that takes the shape of the head, and/or (v) a digital phantom that is derived from a set of magnetic resonance imaging (MRI) or computed tomography (CT) scans of the patient in combination with an electromagnetic field solver (software based) that is able to accurately emulate the experimental measurement. Although the reference scan is absolutely not necessary, in many cases it can help to substantially improve the target stroke (or pathology) signal differentiation and therefore the detection, localization and classification of the same (by subtraction, i.e. ΔS).

In the case of stroke disease, strokes typically occur in one of two types: (i) hemorrhagic or (ii) ischemic. A hemorrhagic stroke is a type of stroke wherein a blood vessel has ruptured, causing uncontrollable bleeding into normal tissue regions, often resulting in substantial intracranial pressure, and leading to partial/complete disability, coma, or death. Similarly dangerous is the ischemic stroke, wherein a small (blood) clot has blocked blood flow to a certain part of the brain. This type of stroke is typically below the spatial resolution of microwave imaging, and is usually not immediately visible and differentiable from normal tissue, even on MRI and CT scans. However, over several hours or days, as a water-based edema forms around the clot occlusion, an ischemic stroke becomes readily detected using microwave imaging technology. The electromagnetic dielectric properties (electrical conductivity and relative permittivity) of an ischemic stroke are known to be approximately 5-20% lower than the head-average dielectric properties of healthy tissue, and consequently provide a somewhat lower microwave imaging contrast in respect to the neighboring healthy tissue, as opposed to a hemorrhagic stroke, which has higher dielectric properties than head-average healthy tissue and therefore yields higher image contrast.

To image such diseases using electromagnetic medical imaging, tomographic imaging methods are used, relying on electromagnetic field solvers based on Maxwell's field equations or variants of the same implemented on a high-speed computer. For any tomographic method to be usable for medical imaging, it is critical to ensure that these solvers can routinely match real-world electromagnetic field-tissue interactions. These electromagnetic field solvers are often called ‘forward’ or ‘inverse’ solvers, and are used in conjunction with the S-parameter measurements as part of the objective function to iteratively optimize a calculated electromagnetic field so that it matches that of the real-world case. There are vast numbers of such algorithms, which are often based on local/global integral or differential tomographic models, often containing Born iterative solvers. Normally the outputs of such optimizations are spatial maps of electrical conductivity and relative permittivity of tissue, often (roughly) indicating the distribution of dielectric properties of the target (e.g., abnormal) tissue, which may or may not be easily visible and differentiated from the surrounding dielectric distribution of normal tissue. In addition, tomographic methods need to solve for orders of magnitude larger number of unknowns than the number of known measurements (e.g., 10,000 unknowns in a 100×100 2D tomographic image, whereas the number of measurements is, for example, only 169, given an array of 14 antennas). Incidentally, tomographic methods suffer from the real possibility that the final imaging result might not converge, despite using the best optimization solvers.

Another common characteristic of the tomographic methods mentioned above is a typically long computational time, even with 2D assumptions (i.e., the subject's anatomy is assumed to be invariant with respect to the z-direction as the third spatial dimension). For example, the computations usually require a wall clock time of several minutes at a minimum, and even hours in cases requiring a high isotropic image spatial resolution (such as 1 mm or 2 mm, for example) to ensure accuracy. Accordingly, a 3D tomographic modelling system may be practically infeasible because the number of voxels increases as the third power of the spatial resolution, and the number of additional electromagnetic tensor field components increases three-fold to a maximum of nine. This would then require substantial investments in supercomputing power (both in terms of the number of CPUs and the amount of RAM), and any tomographic techniques based on, for example, the method of moments (MoM), finite difference time domain (FDTD) or finite element methods (FEM) would require sophisticated parallel computing algorithms, which may not necessarily provide the desired/required computation acceleration (especially for emergency situations of stroke, for instance), despite the large computing resource investment.

Furthermore, radar-based imaging methods require a reasonably accurate dielectric (digital) tissue template of the patient, which is typically unknown due to the large anatomical inter-patient variability, and is not readily available without the additional use of MRI or CT to provide the required morphology for segmentation and digitization.

It is desired, therefore, to provide and apparatus and computer-implemented process for electromagnetic imaging that overcome or alleviate one or more difficulties of the prior art, or to at least provide a useful alternative.

### SUMMARY

In accordance with some embodiments of the present invention, there is provided a computer-implemented process for electromagnetic imaging, the process including the steps of:
accessing scattering data representing at least a two-dimensional array of measurements of electromagnetic wave scattering by internal features of an object, wherein the object is generally symmetrical with respect to a plane of symmetry through the object, and each said measurement represents scattering of electromagnetic waves emitted by a corresponding antenna of an array of antennas disposed about the object as measured by a corresponding antenna of the array of antennas; and
processing the scattering data to generate image data representing a spatial distribution of at least one internal feature of the object, wherein the generation of the image data does not involve tomographic reconstruction but is in accordance with statistical metrics of similarity between pairs of corresponding regions within the object on either side of the plane of symmetry.

The regions within the object may be polygons whose vertices correspond to respective locations of three or more antennas of the array of antennas.

In some embodiments, the step of processing the scattering data includes generating a visibility graph of time series representation of the scattering data. In some embodiments, the accessed scattering data is in the frequency domain, and the step of processing the scattering data includes applying an inverse Fourier transform to the accessed the accessed scattering data to generate the time series of the scattering data.

In some embodiments, the step of processing the scattering data includes selecting a corresponding side of the plane of symmetry with which to associate each statistical metric of similarity.

In some embodiments, the regions of a pair of regions are contained within respective sides of the object with respect to the plane of symmetry, and the selecting is on the basis of similarity metrics between the respective regions and a corresponding region of a reference.

In some embodiments, each of the regions of a pair of regions crosses the plane of symmetry, and the selecting is on the basis of a similarity metric between an upper (and/or lower) portion of each region and a corresponding upper (and/or lower) portion of the other region of the pair of regions, the similarity metrics being computed from scattering parameters representing reflection (e.g., Sii parameters).

In some embodiments, the statistical metrics of similarity are associated with one side of the plane of symmetry selected on the basis of a priori information on the side of the object containing a region of interest having contrasting dielectric properties.

In some embodiments, the step of processing the scattering data includes, for each of a plurality of mesh locations, fusing the statistical metrics of similarity for the mesh location.

In some embodiments, the step of fusing the statistical metrics of similarity includes generating a corresponding expectation value for the statistical metric.

In some embodiments, the object is a human brain, and the at least one internal feature of the object includes a stroke region.

In some embodiments, the computer-implemented process includes classifying the stroke region as being of haemorrhagic or ischemic stroke type in dependence on a comparison of a measure of electromagnetic phase change rate for the stroke region with a corresponding threshold value.

In accordance with some embodiments of the present invention, there is provided at least one computer-readable storage medium having stored thereon at least one of: (i) processor executable instructions and (ii) gate configuration data, which, when executed by at least one processor and/or used to configure gates of a field-programmable gate array, cause the processor and/or the configured gates to execute any one of the above processes.

In accordance with some embodiments of the present invention, there is provided an apparatus for electromagnetic imaging, including:
a memory; and
at least one processor and/or logic components configured to execute any one of the above processes.

In accordance with some embodiments of the present invention, there is provided an apparatus for electromagnetic imaging, including:
an input to receive scattering data representing at least a two-dimensional array of measurements of electromagnetic wave scattering by internal features of an object, wherein the object is generally symmetrical with respect to a plane of symmetry through the object, and each said measurement represents scattering of electromagnetic waves emitted by a corresponding antenna of an array of antennas disposed about the object as measured by a corresponding antenna of the array of antennas; and
an imaging component configured to process the scattering data to generate image data representing a spatial distribution of at least one internal feature of the object, wherein the generation of the image data does not involve tomographic reconstruction but is in accordance with statistical metrics of similarity between pairs of corresponding regions within the object on either side of the plane of symmetry.

In some embodiments, the regions within the object are polygons whose vertices correspond to respective locations of three or more antennas of the array of antennas.

In some embodiments, the generation of the image data includes generating a visibility graph of a time series representation of the scattering data.

In some embodiments, the generation of the image data includes selecting one corresponding side of the object, with respect to the plane of symmetry, with which to associate each statistical metric of similarity.

In some embodiments, the generation of the image data includes, for each of a plurality of mesh locations, fusing the statistical metrics of similarity for the mesh location.

In some embodiments, the object is a human brain, the at least one internal feature of the object includes a stroke region, and the imaging component is configured to classify the stroke region as being of haemorrhagic or ischemic stroke type in dependence on a comparison of a measure of electromagnetic phase change rate for the stroke region with a corresponding threshold value.

Also described herein is a computer-implemented process for electromagnetic imaging, the process including the steps of:
accessing scattering data representing at least a two-dimensional array of measurements of electromagnetic wave scattering by internal features of an object, wherein the object is generally symmetrical with respect to an axis of symmetry through the object, and each said measurement represents scattering of electromagnetic waves emitted by a corresponding antenna of an array of antennas disposed about the object as measured by a corresponding antenna of the array of antennas; and
processing the scattering data to generate image data representing a spatial distribution of at least one internal feature of the object, wherein the generation of the image data does not involve tomographic reconstruction but is in accordance with statistical metrics of similarity between corresponding regions within the object on either side of the axis of symmetry.

Also described herein is an apparatus for electromagnetic imaging, including:
an input to receive scattering data representing at least a two-dimensional array of measurements of electromagnetic wave scattering by internal features of an object, wherein the object is generally symmetrical with respect to an axis of symmetry through the object, and each said measurement represents scattering of electromagnetic waves emitted by a corresponding antenna of an array of antennas disposed about the object as measured by a corresponding antenna of the array of antennas; and
an imaging component configured to process the scattering data to generate image data representing a spatial distribution of at least one internal feature of the object, wherein the generation of the image data does not involve tomographic reconstruction but is in accordance with statistical metrics of similarity between corresponding regions within the object on either side of the axis of symmetry.

### BRIEF DESCRIPTION OF THE DRAWINGS

Some embodiments of the present invention are hereinafter described, by way of example only, with reference to the accompanying drawings, wherein:

FIG. 1 is a block diagram of an apparatus for electromagnetic imaging in accordance with an embodiment of the present invention;

FIG. 2 is a flow diagram of a process for electromagnetic imaging in accordance with an embodiment of the present invention;

FIG. 3 is a schematic plan view of a subject's head and brain surrounded by an array of antennas of the apparatus of FIG. 1, and illustrates symmetric portions of statistical fields (304 and 302) covering two different symmetric areas of the brain;

FIG. 4 is a schematic diagram illustrating comparisons of left-hand side portions of statistical fields (F1L and F2L) with their left-side counterparts in an average medium (F1LA and F2LA, respectively), and right-hand side portions of statistical field (F1R and F2R) with their right-sided counterparts in an average medium (F1RA and F2RA, respectively), for the purpose of determining which side of the statistical fields to activate (i.e., which side of the brain contains a stroke or other anomaly);

FIG. 5 is a schematic diagram illustrating comparisons of upper portions of statistical fields (F2L and F2R) with their lower portion counterparts for the purpose of determining which side of the statistical fields to activate (i.e., which side of the brain contains a stroke or other anomaly);

each of FIGS. 6 to 8 is a set of three pairs of images, each pair comparing an MRI image (left-hand image) to the output of the process (right-hand image) applied to clinical data for a corresponding patient using quadrilateral statistical fields bounded by 4 antennas on each side of the brain in each case, for three different variants or configurations of the process step that decides which side of the brain to activate for each pair of statistical fields (see text for details);

FIGS. 9(a) to (d) are graphs illustrating the successive signal processing steps used to classify stroke type, as follows: (a) a measured phase signal as a function of frequency/sample number, (b) after unwrapping, (c) after linear slope removal, after differentiation (rate of change for every 2 neighbouring points); and

FIG. 10 illustrates the output of the classification process applied to clinical data for eleven tested patients (see text for details).

### DETAILED DESCRIPTION

Embodiments of the present invention include an apparatus and computer-implemented process for efficient and rapid electromagnetic imaging of one or more internal features of an object and avoiding any form of tomographic reconstruction. The process is generally applicable to any object that is generally symmetrical with respect to an axis of symmetry through the object, and to image one or more internal features that are not themselves symmetrically located or distributed with respect to the axis of symmetry.

Accordingly, the process described herein includes the steps of generating or otherwise accessing scattering data representing at least a two-dimensional array of measurements of electromagnetic wave scattering by internal features of the object. Each such measurement represents the scattering of electromagnetic waves emitted by a corresponding antenna of an array of antennas disposed about the object, as measured by a corresponding antenna of the array of antennas. The scattering data is processed to generate data (referred to herein for convenience of reference as “image data”, notwithstanding that it is not essential that an “image” as such is ever generated or displayed) representing a spatial distribution of at least one internal feature of the object. As indicated above, the generation of the image data does not require or involve tomographic reconstruction. Rather, it is generated in accordance with statistical metrics of similarity between corresponding regions within the object on either side of the axis of symmetry.

Although embodiments of the present invention are described below in the context of imaging anomalies such as stroke in the human brain, it should be understood that the described apparatus and process can equally be applied to image internal features of any object (whether biological or otherwise), provided that the object is generally or at least approximately symmetrical with respect to an axis of symmetry through the object, and that the internal features themselves are not symmetrically located with respect to the axis of symmetry.

In the context of detecting anomalies in the human brain, the process generally relies on the assumption that the two halves of the brain are highly similar in structure and composition, such that any significant asymmetry in electromagnetic interactions with respect to the brain's axis of symmetry is indicative of an anomaly. The process not only identifies such anomalies, but is also able to indicate their specific location and severity within the brain. The ability to do this essentially in real-time is extremely important for the time-critical detection and assessment of strokes and injuries in the brain, where permanent brain injury, disability and death can result if treatment is not performed in a timely manner.

More specifically, the apparatus and process of the described embodiments exploit multivariate statistics to compute measures of statistical difference between nominally symmetrical regions of the brain. In the described embodiments, these symmetrical regions are in the form of polygons whose vertices correspond to the locations of at least 3 antennas on each side of the axis of symmetry. After all of the statistical measures, also referred to herein as “statistical fields”, have been constructed and computed, their values are fused to generate corresponding probability values.

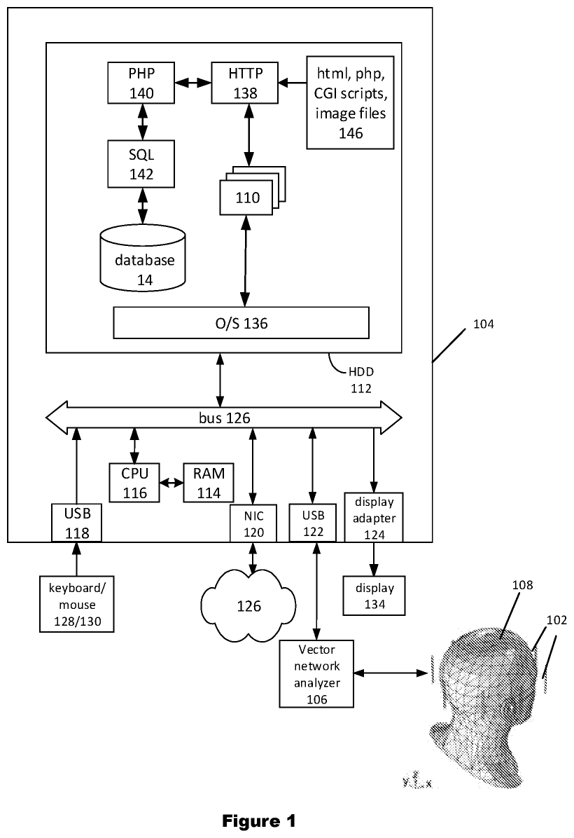

As shown in FIG. 1, an electromagnetic imaging apparatus includes an array of microwave antennas 102 coupled to a data processing component 104 via a vector network analyzer (VNA) or transceiver 106.

The array of microwave antennas 102 is arranged to receive the head 108 of a patient whose brain is to be imaged, as shown, so that each antenna of the array can be selectively energised to radiate electromagnetic waves or signals of microwave frequency into and through the subject's head 108 to be scattered, and the corresponding scattered signals detected by all of the antennas 102 of the array, including the antenna that transmitted the corresponding signal.

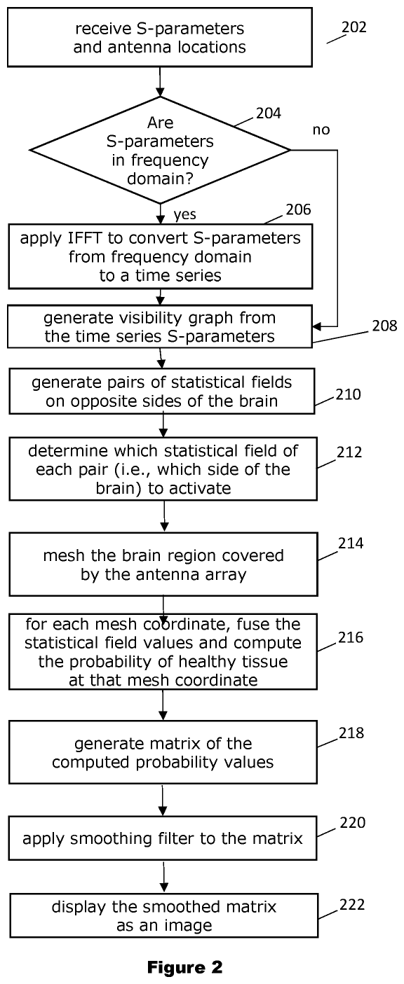

As will be apparent to those skilled in the art, the vector network analyser (VNA) 106 energises the antennas as described above, and records the corresponding signals from the antennas as data (referred to herein as ‘scattering’ data) representing the amplitudes and phases of the scattered microwaves in a form that is known in the art as “scattering parameters” or “S-parameters”. The VNA 106 sends this data to the data processing component 104, which executes an electromagnetic imaging process, as shown in FIG. 2, to generate information on internal features of the imaged object (e.g., brain clots, bleeding sites, and other features) that can (but need not) be used to generate images of those features. In the described embodiments, a VNA that has a large dynamic range of more than 100 dB and a noise floor below −100 dBm can be used to activate the antennas 102 to transmit electromagnetic signals across the frequency band of 0.5 to 4 GHz and receive the scattered signals from those antennas 102.

Although the data processing component 104 of the described embodiments is in the form of a computer, this need not be the case in other embodiments. As shown in FIG. 1, the electromagnetic imaging apparatus of the described embodiments is a 64-bit Intel Architecture computer system, and the electromagnetic imaging processes executed by the electromagnetic imaging apparatus are implemented as programming instructions of one or more software components 110 stored on non-volatile (e.g., hard disk or solid-state drive) storage 112 associated with the computer system. However, it will be apparent that at least parts of these processes could alternatively be implemented in one or more other forms, for example as configuration data of a field-programmable gate array (FPGA), or as one or more dedicated hardware components, such as application-specific integrated circuits (ASICs), or as any combination of such forms.

The stroke monitoring apparatus includes random access memory (RAM) 114, at least one processor 116, and external interfaces 118, 120, 122, 124, all interconnected by a bus 126. The external interfaces include a network interface connector (NIC) 120 which connects the electromagnetic imaging apparatus to a communications network such as the Internet 126, and universal serial bus (USB) interfaces 118, 122, at least one of which may be connected to a keyboard 128 and a pointing device such as a mouse 130, and a display adapter 124, which may be connected to a display device such as an LCD panel display 134.

The electromagnetic imaging apparatus also includes an operating system 136 such as Linux or Microsoft Windows, and in some embodiments includes additional software components 138 to 144, including web server software 138 such as Apache, available at http://www.apache.org, scripting language support 140 such as PHP, available at http://www.php.net, or Microsoft ASP, and structured query language (SQL) support 142 such as MySQL, available from http://www.mysql.com, which allows data to be stored in and retrieved from an SQL database 144.

Together, the web server 130, scripting language module 132, and SQL module 134, in combination with the electromagnetic imaging software components 110 and support files 146 (typically including html, php and/or CGI scripts and associated image files), provide the electromagnetic imaging apparatus with the general ability to allow remote users with standard computing devices equipped with standard web browser software to access the electromagnetic imaging apparatus and in particular to determine (and typically view a visual representation of) the location(s) of a stroke or other form of brain injury, and optionally to monitor its progress over time. For the sake of simplicity, the electromagnetic imaging apparatus and process are described herein in the context of a single array of antennas lying in a transverse plane that passes through the subject's brain and stroke region (i.e., to provide 2D localisation of the stroke in the transverse plane), although the same steps apply to “3D” cases in which there are two or more layers of antennas available to provide three-dimensional localization.

As shown in FIG. 2, a process for electromagnetic imaging of a brain anomaly begins by receiving (e.g., from the VNA 101) or otherwise accessing (e.g., from storage) at step 202 scattering data, in the described embodiments being in the form of “S-parameters” representing at least a two-dimensional array of measurements of electromagnetic wave scattering by internal features of a subject's brain, as described above. In the described embodiments, an array of 16 antennas 102 is used, resulting in a 16×16 array or matrix of measurements in either the frequency domain or the time domain.

At step 204, a test is performed to determine whether the S-parameters are in the frequency domain, and, if so, then at step 206 an inverse fast Fourier transform (“IFFT”) is applied to the S-parameters individually to convert them to the time domain. Although other embodiments may directly process the time domain signals, the inventors have found that noise can be reduced if the time domain measurements are converted to network form. Accordingly, at step 208, a network (i.e., graph) representation of the time domain measurements is generated in a form referred to as a ‘visibility graph’ using the method described in Lacasa, Lucas, et al., “From time series to complex networks: The visibility graph”, Proceedings of the National Academy of Sciences 105.13 (2008): 4972-4975.

The human brain is of course generally symmetrical with respect to the median plane, characterized (in the two-dimensional transverse plane of the antenna array) by an axis of symmetry between the left and right halves of the brain, and each measurement represents scattering of electromagnetic waves emitted by a corresponding antenna of the array of antennas disposed about the object as measured by a corresponding antenna of the array of antennas.

Consequently, in the absence of any anomalies in the subject's brain, the signals transmitted and received by an arbitrary pair of the antennas 102 on one side of the subject's head should be equal to the signals of the corresponding pair of the antennas 102 on the symmetrically opposite side of the subject's head. The actual level of similarity between a pair of such symmetrical measurements can be quantified using a statistical index. In the described embodiments, a multivariate statistical index is used to statistically compare two mutually symmetrical (and symmetrically located with respect to the brain's axis of symmetry) regions of the brain, where each region is a simple polygon or n-gon defined by at least three (n>3) vertices corresponding to respective antenna locations. Each such region and its associated similarity index are collectively referred to herein as a ‘statistical field’, noting that in the present context this term has a different meaning to its meaning in the fields of polymer physics and biophysics. However, for convenience of description, sometimes each component of a statistical field may also be referred to herein as a statistical field, notwithstanding that formally a statistical field is defined by both components.

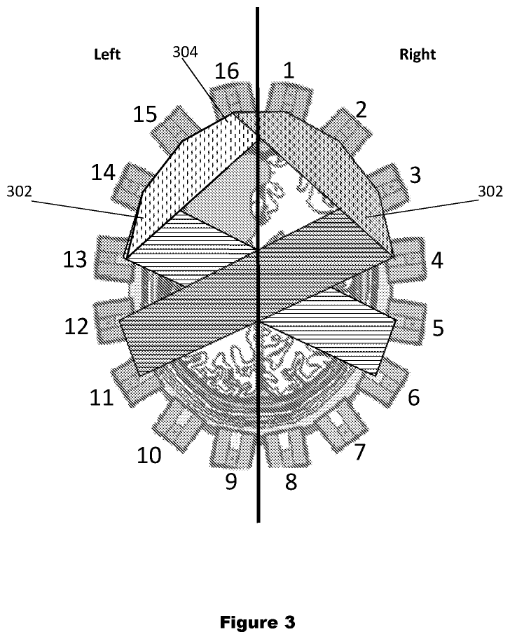

At step 210, the statistical fields are generated. In any one embodiment, the statistical fields may have the same shape or different shapes. The simplest statistical field is one bounded by only three antennas, and consequently has a triangular shape, which can be of any type (i.e., equilateral, right, isosceles, acute, obtuse or scalene). Examples of statistical fields defined by the locations of 4 and 5 antennas (i.e., tetragons 302 and pentagons 304, respectively) are shown in FIG. 3.

Any pair of mutually symmetrical and symmetrically located regions of the brain can be compared (in the sense of differences of electromagnetic signal propagation and scattering within them) by means of a multivariate statistical index, and to this end the apparatus and processes of the described embodiments use the distance correlation (“dCorr”) index described in Székely, Gábor J., Maria L. Rizzo, and Nail K. Bakirov, “Measuring and testing dependence by correlation of distances”, The annals of statistics 35.6 (2007): 2769-2794 (“Székely”). However, it will be apparent to those skilled in the art that other statistical measures of similarity (e.g., Mutual Information or Person coefficients) may be used in other embodiments.

Distance correlation statistics is a class of energy statistics based on distances which measures the dependencies of random variables of arbitrary size and distributions, and as such can be also employed as an index of dependence.

As described in Székely:

Let X=[x₁, . . . , xₚ]^{T} and Y=[y₁, . . . , y_{q}]^{T} be two random vectors with finite first moments, i.e. E(∥X∥+∥Y∥)<∞. Let also X¹, . . . , X^{N} be N independent and identically distributed (“i.i.d.”) realizations of X, and Y¹, . . . , Y^{N} the corresponding i.i.d. realizations of Y. Then dCorr is defined as:

$${R}_{N}^{*} ( x , y ) = \frac{{v}_{N}^{*} ( x , y )}{\sqrt{{v}_{N}^{*} ( x ) {v}_{N}^{*} ( y )}} , {v}_{N}^{*} ( x , y ) = \frac{1}{{N}^{2}} {\sum }_{k , l = 1}^{N} {A}_{kl} {B}_{kl} \\ where {A}_{kl} = {a}_{kl} - \overline{{a}_{k} .} - \overline{{a}_{. l}} + \overline{a …} , {B}_{kl} = {b}_{kl} - \overline{{b}_{k} .} - \overline{{b}_{. l}} + \overline{b …} , \\ {a}_{kl} = ‖ {x}^{k} - {x}^{l} ‖ , {b}_{kl} = ‖ {y}^{k} - {y}^{l} ‖ , \overline{{a}_{k} .} = \frac{1}{N} {\sum }_{l = 1}^{N} {a}_{kl} , \\ \overline{{a}_{. l}} = \frac{1}{N} {\sum }_{k = 1}^{N} a - kl , \overline{a …} = \frac{1}{{N}_{2}} {\sum }_{k , l = 1}^{N} {a}_{kl} , \\ \overline{b .} = \frac{1}{N} {\sum }_{l = 1}^{N} {b}_{kl} , \overline{{b}_{. l}} = \frac{1}{N} {\sum }_{k = 1}^{N} {b}_{kl} and \overline{b …} = \frac{1}{{N}_{2}} {\sum }_{k , l = 1}^{N} {b}_{kl} . \qquad ( 3.1 )$$

One of the properties of the dCorr index (R_{N}*) (see Theorem 3 of Székely) is that R_{N}*(X, Y)=1 if there exists a vector b, and a nonzero real number k and an orthogonal matrix C such that:

Y=kXC+b, (3.2).

Accordingly, if signals comprising a statistical field on the left side of the brain are denoted by X, and signals comprising the symmetrically equivalent statistical field on the right side are denoted by Y, then assuming the electromagnetic signals measured on one side of the head are equal to their symmetrical counterparts, then equation 3.2 becomes Y=X for k=1 and b=0. This implies that in case of abnormality (e.g., a stroke region in one of the statistical fields) this property will not hold, and consequently the dCorr index will have a value less than 1. The larger the stroke region, the lower will be the statistical similarity between the signals passing through the stroke region and its symmetrical counterpart, and consequently the lower the dCorr index value. It should be noted that the transmission signals (Sᵢⱼ) from sensors that are far from each other provide more information about the deep targets inside the brain, while the signals from immediately adjacent antennas mainly carry information from shallow brain areas and outer layer tissues such as skin and skull. For this reason, when computing the statistical fields, signals of immediately adjacent antennas are not included in the calculations.

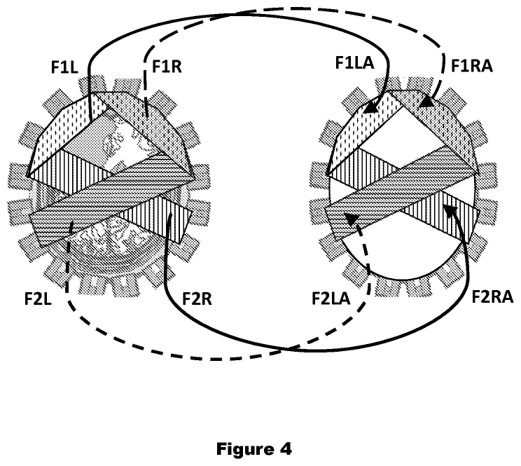

However, because the intensity of a statistical field is a metric of its similarity to its corresponding (i.e., mirror-image counterpart) statistical field on the other side of the brain, in the described context of anomaly detection a decision needs to be made at step 212 as to which side of the brain contains the anomaly. The statistical field containing the anomaly is said to be ‘active’, and only the active statistical fields are used to compute an overall field intensity for the corresponding mesh point or coordinates (or “pixel” where the overall field intensity is considered to constitute an image of a brain anomaly).

Where prior information indicating which side of the brain contains the stroke (or other anomaly) is not available, the process computes the similarity of each statistical field (e.g., the field on the left side of the brain) to the same region of a reference (e.g., a reference medium having the average electromagnetic properties of the human brain), as illustrated in FIG. 4. The statistical field with the smallest value of similarity (i.e., the lowest dCorr index) relative to the reference is selected as the side containing the stroke.

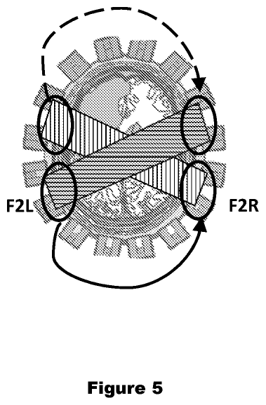

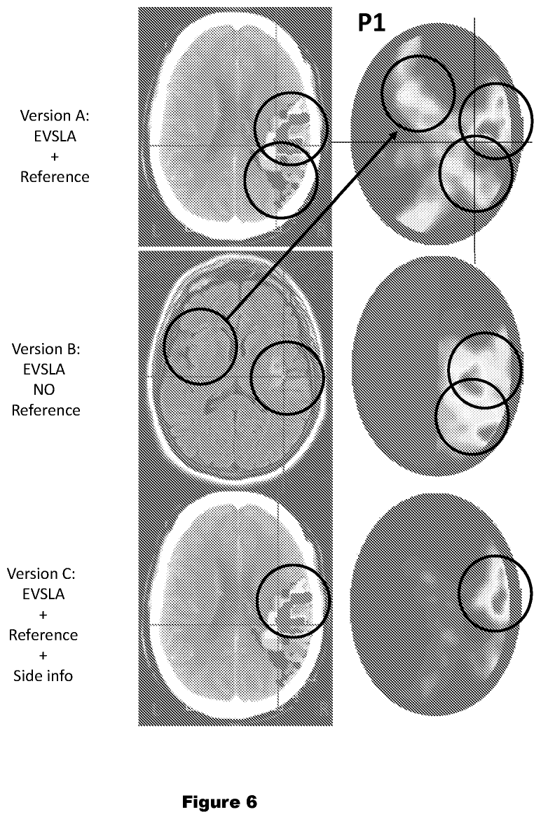

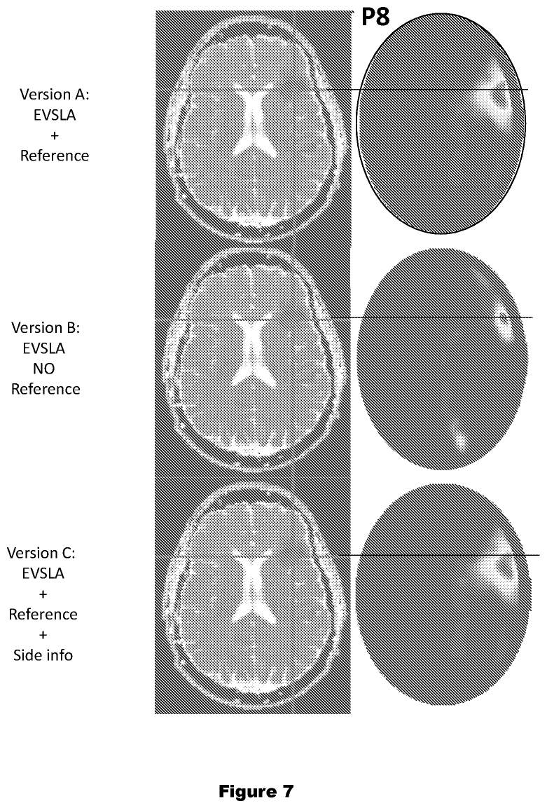

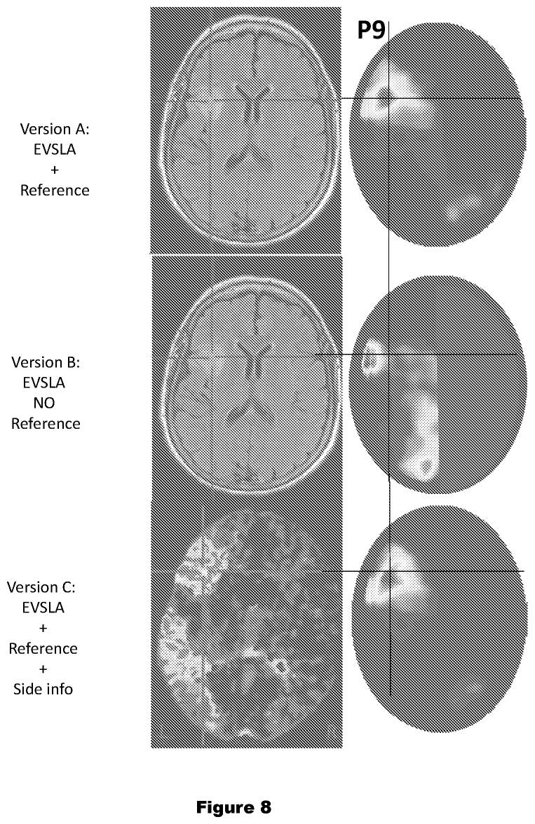

If, however, stroke side information can be provided by doctors, then it can serve as a priori information for the process to decide which side of the brain contains the anomaly, without requiring reference signals. In particular, for statistical fields for which the right and left counterparts are each completely contained within the corresponding half of the brain, prior information on stroke side is sufficient to activate the correct side. However, in cases where the statistical fields extend over both halves of the brain, for example as illustrated in FIG. 5, the prior information is insufficient because it does not provide enough information to confirm which of the two statistical fields is active, and additional information is required. In order to address this difficulty, the process computes the similarity (using the dCorr index) by using reflection signals (Sᵢᵢ) of the antennas at the upper left and upper right vertices of the statistical fields, and separately for the lower left and right vertices, as illustrated in FIG. 5. If the difference between the upper left and upper right vertices is bigger than for the lower left and right vertices, then the polygon whose upper vertex is on the injured side will be selected. Otherwise, the polygon whose lower vertex is on the injured side will be selected.

Multiple overlapping statistical fields covering different regions of the brain are generated so that any given location within the brain is contained within multiple statistical fields. To compute the value or ‘intensity’ of the overall statistical field at a given point inside the subject's brain resulting from that point being included within multiple overlapping statistical fields, the imaging domain surrounded by the antenna array is represented as a mesh. Each point of the mesh can be treated individually, and the integral intensity of all statistical fields encompassing that point (or pixel) can be computed by combining or ‘fusing’ their values, as described below.

After the process decides which side of the brain to activate for all generated pairs of statistical fields, a mesh covering the brain region within the antenna array is generated at step 214, and at step 216, the similarity values of each mesh coordinate are combined or ‘fused’. The simplest way to fuse the information is to sum all of the values of the statistical fields encompassing the mesh coordinate (or pixel), as follows:

dCorr(g)=Σᵢ₌₁^{N}^{ₚ}dCorr(i), (3.3)

where Nₚ denotes the total number of generated statistical fields encompassing pixel g, g=1, . . . , N_{G}. Although it is the simplest method of combining values, it is not the most useful because it does not allow mathematical interpretation of the results.

Accordingly, in the described embodiments the process fuses the information by computing the expected value of the similarity metric for each pixel individually. Assuming that the dCorr index values for the statistical fields encompassing a given mesh coordinate are drawn from some (but the same) probability distribution, then the expected value of the probability distribution for a given pixel can be estimated by the mean, according to:

$$E [ dCorr ( g ) ] = \frac{1}{{N}_{p}} {\sum }_{i = 1}^{{N}_{p}} dCorr ( i ) . \qquad ( 3.3 )$$

Considering that the size of the population of statistical fields Nₚ is a design parameter and thus can take any value, it is helpful to recall The Central Limit Theorem, which states that, given a sufficiently large sample size from a population with a finite variance, the mean of all samples from the same population will be approximately equal to the mean of the population.

After the field intensities are computed for all pixels individually at step 216, a matrix of the computed expected values is generated at step 218. In the final step, before pixel probability values are assigned to a colour scale to visualize the results (i.e., stroke localization), a smoothing filter is applied along the x-axis and the y-axis at step 220. This improves visualization of the results by compensating for noise in some pixels. In the described embodiments, the smoothing filter calculates average values in a sliding window of width W pixels. To increase visual contrast, exponentiation of a desired degree can optionally be applied to the matrix of expected values.

### EXAMPLES

FIGS. 6 to 8 show results obtained from clinical data collected with the 16 antennas of the apparatus operating in a frequency band of 0.5-1.5 GHz, although the images shown were generated for a frequency band of 0.7-1.598 GHz. The process was applied to the same set of scattering parameters, but with three different configurations for deciding which statistical fields to activate, as follows:
Version A. using only a reference as described above;
Version B. using only prior information on stroke side; and
Version C. using a combination of a reference and prior information, where the prior information is used for activation when each of the pair of statistical fields is completely contained within a corresponding side of the brain (i.e., they do not cross the axis of symmetry), and the reference is used when each statistical field of a pair crosses the axis of symmetry.

The results were obtained for an initial domain discretization of 2 mm, the first 100 points of the network signals of a statistical field, a sliding window of width W=4, and exponentiation of 5^{th} degree in the final step. To obtain a finer image representation, the generated image matrix values were interpolated linearly with a step size of 10-3.

The results in FIGS. 6 to 8 demonstrate the ability of the apparatus and process described herein to successfully localise stroke targets in all three configurations of the process, with relatively minor differences between them. As shown in FIG. 6, for one patient version A of the process (upper images) was successful at detecting both old and new stroke regions, whereas the other two versions of the process (middle and lower images) detected only the new stroke region Similar results can be observed for the other two patients, as shown in FIGS. 7 and 8, respectively. However, Version B of the process relies on computing similarity metrics using non-transmission data (it uses reflection coefficients known in the art as “Sii parameters”), and consequently might be less accurate for detection of deep and small stroke targets.

It is important to note that the spatial resolution of the described apparatus and process strongly depends on the number of antennas in the array arranged around the subject's head. However, the number of antennas is limited by the available space around the head, the size of each antenna and their mutual coupling. Consequently, strokes with dimensions less than the distance between two neighbouring antennas might be captured in a statistical sense but the spatial dimensions might possibly not be visualized accurately.

When implemented as Matlab modules on a 3.7 GHz Intel Xeon W-2145 workstation with 64 GB of RAM, the average execution time of the process was 7 s, including generating 70 statistical fields, fusing similarity values and estimating the probability for each pixel, deciding the stroke side, and visualizing the results. If the stroke side is known a priori, then the execution time is halved. In addition, recoding the process in a low-level language such as C could reduce the execution time by an order of magnitude.

### Stroke Classification

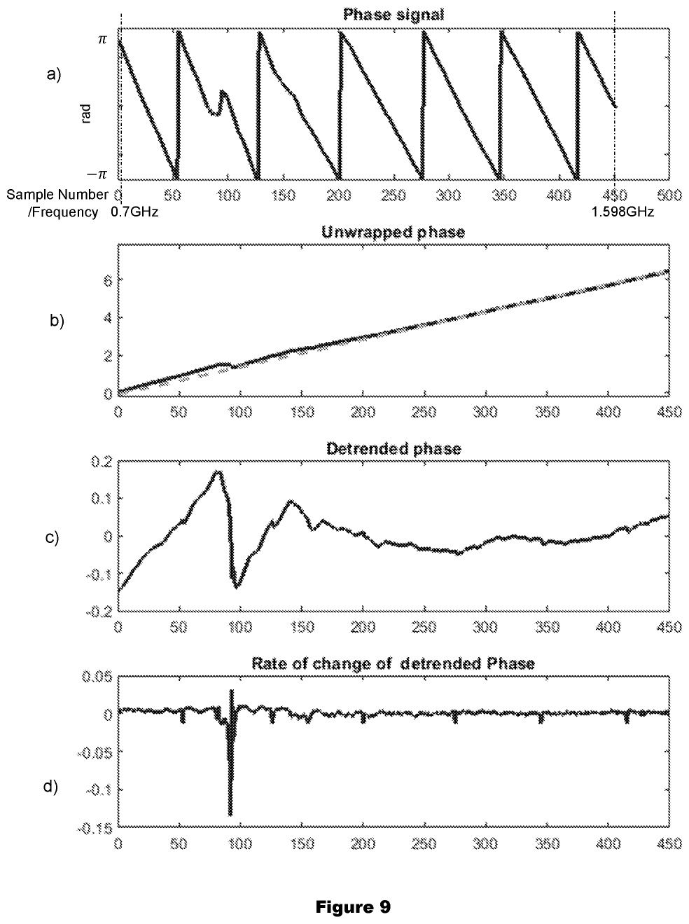

Stroke classification relies on changes in phase due to changes in the dielectric properties of the imaging domain. The phase signal of each Sᵢⱼ is obtained by determining its imaginary and real parts over the considered frequency band. FIG. 9(a) is a graph showing the phase of a signal S_{14,5} over 450 frequency samples in the frequency range 0.7-1.598 GHz. For each antenna pair, i.e., transmission signal Sij, the following steps are performed:
Step 1. The phase signal is scaled to the 0-1 range and then ‘unwrapped’ to provide a corresponding unwrapped phase signal ranging from 0 to kπ, where k>0 and equals total number of 180° jumps in phase in the considered range. FIG. 9(b) is a graph of the unwrapped phase signals of FIG. 9(a).
Step 2. The linear trend representing the slope which characterizes the original phase signal unwrapped (denoted as dashed grey in FIG. 9(b)) is subtracted from the signal obtained in Step 1. (see FIG. 9(c)).
Step 3. For the signal obtained in Step 2, the rate of change of the signal is computed for every 2 neighbouring points. (see FIG. 9(d))
Step 4. Compute the standard deviation of the rates of change computed in Step 3.

Once these steps have been applied to all transmission signals, the average value of the standard deviations obtained in Step 4 is calculated.

Determining whether the resulting average value is above or below a threshold T discriminates haemorrhagic (bleeding) from ischemic (clot) stroke; i.e., determines the stroke type. Given a large number of patients (e.g., 100 patents, where at least 30-40% of them have one stroke type and the remaining have the other stroke type) would enable the classification result to be given with a high level of confidence. For example, the process can display to a user an output message such as: ‘The case X is stroke type Y with a confidence level of 95%’.

In addition, having more samples enables the use of Machine Learning clustering to learn cluster centroids and cluster ranges for subsequent stroke classification. Another alternative is to employ any ML classification method (e.g., Support Vector Machine (SVM), Nearest neighbours (NN), Logistic Regression, (Deep) Neural Networks (DNN), Naïve Bayes) to learn the hyperplane that separates two stroke types based on the computed rate of phase change.

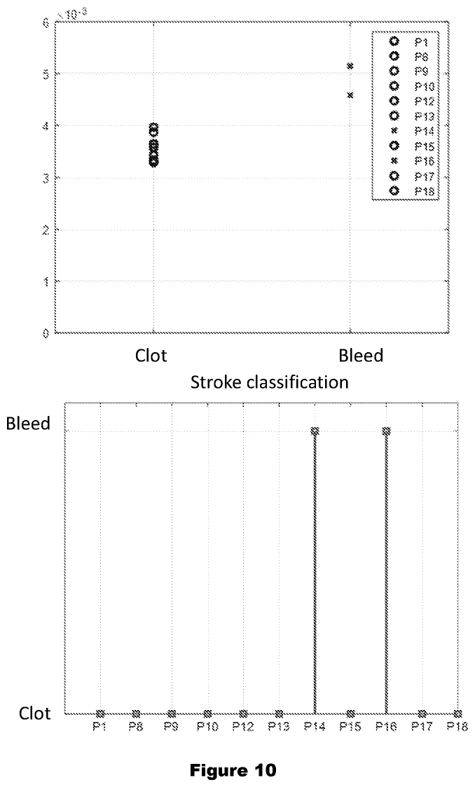

FIG. 10 shows results obtained from clinical data collected with the 16 antennas of the apparatus operating in a 0.7-1.598 GHz frequency range for 11 patients, two with bleeding and the reminder with a blood clot. The classification process described above was applied to scattering data obtained for a frequency band of 0.8960-1.096 GHz, which has been identified as the range with the biggest difference in phase for the aforementioned two stroke types. FIG. 10 (top) illustrates the distribution of the average value of the standard deviation for the computed rates of phase change, showing that the values for the two stroke types do not overlap. Accordingly, given a threshold value T=0.004, the process correctly determines the stroke type, as shown in the lower part of FIG. 10. These classification results are in agreement with the ground truth provided by doctors.

The results described above confirm that the apparatus and process described herein are capable of detecting stroke targets and determining their locations and dimensions within the brain. In the case of stroke targets that are at least as large as the Euclidian distance between two neighbouring antennas, they are capable of determining the size and indicating the shape of the stroke region. In the case of small targets that are significantly smaller than the Euclidian distance between two neighbouring antennas (approximately 2 or more times smaller), they may localize stroke regions correctly, but the stroke dimensions might not be accurate. This is not surprising because the spatial resolution is mainly determined by the number of the antennas in the antenna array. Increasing the number of available signals increases the localization resolution, and hence the precision of the determined target size.

The apparatus and process described herein therefore:
are suitable for localization of both types of stroke, located anywhere in the head except along the symmetry plane;
are capable of differentiating between ischemic and haemorrhagic stroke types;
do not require a priori knowledge of the shape or dielectric properties of the imaging domain;
can detect and locate big and small targets;
do not require a reference (e.g., data collected in an average medium) if the stroke side is known (left or right side of the brain); and
have a typical execution time of less than a minute on standard personal computer hardware at the time of writing, which makes them suitable for real-time applications.

The main requirements are related to antenna array positioning, namely:
symmetrical positioning of the antenna array with respect to the major axis of the brain (or other object) to ensure the same distance from the major axis of the object; and
any tilt of the antenna array on both sides of the brain with respect to the vertical plane passing through the major axis of the brain to be the same.

Many modifications will be apparent to those skilled in the art without departing from the scope of the present invention.

1. A computer-implemented process for electromagnetic imaging, the process including the steps of:
accessing scattering data representing at least a two-dimensional array of measurements of electromagnetic wave scattering by internal features of an object, wherein the object is generally symmetrical with respect to a plane of symmetry through the object, and each said measurement represents scattering of electromagnetic waves emitted by a corresponding antenna of an array of antennas disposed about the object as measured by a corresponding antenna of the array of antennas; and
processing the scattering data to generate image data representing a spatial distribution of at least one internal feature of the object, wherein the generation of the image data does not involve tomographic reconstruction but is in accordance with statistical metrics of similarity between pairs of corresponding regions within the object on either side of the plane of symmetry.

2. The computer-implemented process of claim 1, wherein the regions within the object are polygons whose vertices correspond to respective locations of three or more antennas of the array of antennas.

3. The computer-implemented process of claim 1, wherein the step of processing the scattering data includes generating a visibility graph of a time series representation of the scattering data.

4. The computer-implemented process of claim 3, wherein the accessed scattering data is in the frequency domain, and the step of processing the scattering data includes applying an inverse Fourier transform to the accessed the accessed scattering data to generate the time series scattering data.

5. The computer-implemented process of claim 1, wherein the step of processing the scattering data includes selecting a corresponding side of the plane of symmetry with which to associate each statistical metric of similarity.

6. The computer-implemented process of claim 5, wherein the regions of a pair of regions are contained within respective sides of the object with respect to the plane of symmetry, and the selecting is on the basis of similarity metrics between the respective regions and a corresponding region of a reference.

7. The computer-implemented process of claim 5, wherein each of the regions of a pair of regions crosses the plane of symmetry, and the selecting is on the basis of a similarity metric between an upper (and/or lower) portion of each region and a corresponding upper (and/or lower) portion of the other region of the pair of regions, the similarity metrics being computed from scattering parameters representing reflection (e.g., Sii parameters).

8. The computer-implemented process of claim 5, wherein the statistical metrics of similarity are associated with one side of the plane of symmetry selected on the basis of a priori information on the side of the object containing a region of interest having contrasting dielectric properties.

9. The computer-implemented process of claim 1, wherein the step of processing the scattering data includes, for each of a plurality of mesh locations, fusing the statistical metrics of similarity for the mesh location.

10. The computer-implemented process of claim 9, wherein the step of fusing the statistical metrics of similarity includes generating a corresponding expectation value for the statistical metric.

11. The computer-implemented process of claim 1, wherein the object is a human brain, and the at least one internal feature of the object includes a stroke region.

12. The computer-implemented process of claim 11, including classifying the stroke region as being of haemorrhagic or ischemic stroke type in dependence on a comparison of a measure of electromagnetic phase change rate for the stroke region with a corresponding threshold value.

13. At least one computer-readable storage medium having stored thereon at least one of: (i) processor executable instructions and (ii) gate configuration data, which, when executed by at least one processor and/or used to configure gates of a field-programmable gate array, cause the processor and/or the configured gates to execute the process of claim 1.

14. An apparatus for electromagnetic imaging, including:
a memory; and
at least one processor and/or logic components configured to execute the process of claim 1.

15. An apparatus for electromagnetic imaging, including:
an input to receive scattering data representing at least a two-dimensional array of measurements of electromagnetic wave scattering by internal features of an object, wherein the object is generally symmetrical with respect to a plane of symmetry through the object, and each said measurement represents scattering of electromagnetic waves emitted by a corresponding antenna of an array of antennas disposed about the object as measured by a corresponding antenna of the array of antennas; and
an imaging component configured to process the scattering data to generate image data representing a spatial distribution of at least one internal feature of the object, wherein the generation of the image data does not involve tomographic reconstruction but is in accordance with statistical metrics of similarity between pairs of corresponding regions within the object on either side of the plane of symmetry.

16. The apparatus of claim 15, wherein the regions within the object are polygons whose vertices correspond to respective locations of three or more antennas of the array of antennas.

17. The apparatus of claim 15, wherein the generation of the image data includes generating a visibility graph of a time series representation of the scattering data.

18. The apparatus of claims 15, wherein the generation of the image data includes selecting one corresponding side of the object, with respect to the plane of symmetry, with which to associate each statistical metric of similarity.

19. The apparatus of claim 15, wherein the generation of the image data includes, for each of a plurality of mesh locations, fusing the statistical metrics of similarity for the mesh location.

20. The apparatus of claim 15, wherein the object is a human brain, the at least one internal feature of the object includes a stroke region, and the imaging component is configured to classify the stroke region as being of haemorrhagic or ischemic stroke type in dependence on a comparison of a measure of electromagnetic phase change rate for the stroke region with a corresponding threshold value.
